(19) United States
(12) Patent Application Publication
(10) Pub. No.: US 2011/0201956 A1
(43) Pub. Date: Aug. 18, 2011

(54) DIRECT LUNG SENSOR SYSTEMS, METHODS, AND APPARATUSES

(76) Inventors: Clifton A. Alferness, Port Orchard, WA (US); Steven C. Springmeyer, Bellevue, WA (US); Brandon James Shuman, Kirkland, WA (US); William A. Sirokman, Kirkland, WA (US); Hugo X. Gonzalez, Woodinville, WA (US); Clinton L. Finger, Bellevue, WA (US)

(21) Appl. No.: 12/913,257

(22) Filed: Oct. 27, 2010

Related U.S. Application Data
(63) Continuation of application No. PCT/US2009/042422, filed on Apr. 30, 2009.
(60) Provisional application No. 61/049,573, filed on May 1, 2008.
(60) Provisional application No. 61/160,248, filed on Mar. 13, 2009.

(51) Int. Cl. A61B 5/087; A61B 5/08; A61M 16/00

(52) U.S. Cl. 600/532; 600/538; 128/200.24; 600/529

(57) ABSTRACT

Devices, systems, and methods for diagnosing physiological parameters of the lungs and treating associated medical conditions are disclosed herein. In particular, certain embodiments permit detection of air flow in lung passageways, air leaks, gas concentration (in particular oxygen), and temperature measurements. Measurements obtained using the devices, systems, and methods disclosed herein may also be used to determine optimal treatment sites for medical conditions such as emphysema, COPD, or lung volume reduction.

### CROSS REFERENCE TO RELATED APPLICATIONS

This application claims priority under 35 U.S.C. § 119(e) to U.S. Provisional Application No. 61/049573, filed May 1, 2008. This application also claims priority under 35 U.S.C. § 119(e) to U.S. Provisional Application No. 61/160248, filed Mar. 13, 2009. Both of these applications are hereby incorporated by reference in their entirety.

### BACKGROUND OF THE INVENTION

1. Field of the Invention,

The present invention relates generally to medical methods, systems, and devices designed and used to detect physiological characteristics within a lumen. More particularly, certain features, aspects or embodiments of the present invention relate to methods, systems and devices for performing diagnostic testing, evaluation or monitoring within or directly adjacent individual sections, subsections, segments, or areas of a lung of a patient.

2. Description of the Related Art

Chronic Obstructive Pulmonary Disease (COPD) has become a major cause of morbidity and mortality in the United States over the last three decades. COPD is characterized by the presence of airflow obstruction due to chronic bronchitis or emphysema. The airflow obstruction in COPD is due largely to structural abnormalities in the smaller airways. Important causes are inflammation, fibrosis, goblet cell metaplasia, and smooth muscle hypertrophy in terminal bronchioles.

COPD affects the patient's whole life. It has three main symptoms: cough; breathlessness; and wheeze. At first, breathlessness may be noticed when running for a bus, digging in the garden, or walking up hills. Later, it may be noticed when simply walking in the kitchen. Over time, it may occur with less and less effort until it is present all of the time.

COPD, and in particular emphysema, is usually not evenly distributed through the lung. To address this non-even distribution, a treatment has been developed that can specifically target the non-uniform condition by selectively placing valves in the bronchial passageways. Examples of such valves are described in U.S. Pat. No. 6,293,951 and other patents and published applications.

Moreover, lungs can develop air leaks as a result of incomplete sealing of the pleura following a lung surgical procedure, as a result of tears that occur as a result of pleural adhesions or as a result of tears that occur as a result of sudden pressure differentials. The leaks also can form in portions of the lungs that have been weakened by lung diseases, such as emphysema, for example. Identification of the specific location of the leaks within the lung can be difficult and, therefore, treatment of a persistent leak can be difficult.

Moreover, the lungs comprise a plurality of bronchopulmonary compartments. A double layer of infolded reflections of visceral pleura, which are called fissures, generally separate the bronchoplumonary compartments. The fissures are typically impermeable and the lung compartments receive and expel air only through the upper airways that open into the compartments. While the compartments within particular lung lobules communicate with each other through certain collateral pathways, such pathways are generally not thought to pass through the impermeable fissures that separate the lung compartments.

Studies have shown that the fissures are not always complete, and therefore, the lobular regions of the lungs may be connected and provide a pathway for collateral airflow. Accordingly, a collateral drift of air is believed to pass from one pulmonary segment into the next, and this phenomenon is known generally as collateral ventilation. The presence of collateral pathways between lung compartments is markedly increased in emphysema patients. Further, the presence of collateral pathways in the lungs may make treatments for chronic obstructive pulmonary disease (“COPD”), such as Endobronchial Volume Reduction (“EVR”), less effective. The presence of collateral pathways may make a desired volume reduction difficult due to air being drawn in from neighboring lung compartments via the collateral channels.

### SUMMARY OF THE INVENTION

A need exists for an efficient system and manner to locate regions of the lung that would benefit from the placement of the treatment devices. In other words, a system is desired that would guide placement of the devices. The devices could be used to treat COPD, air leaks, collateral ventilation or the like.

In addition, a direct, accurate, simple and minimally invasive method for accurately assessing and diagnosing the health and ventilation of specific portions of the lung is desirable.

Thus, certain features, aspects and advantages of various embodiments the present invention provide systems and methods for determining and assessing physiological parameters of the lungs by sensing the physiological parameters directly within the lung.

In certain embodiments of the present invention, systems and methods for targeted or minimally-invasive evaluation of physiological parameters of the lungs are provided. Particular locations in the lung may be examined locally and assessed by means of various sensors disposed on a catheter. In certain embodiments, this catheter may be delivered through a bronchoscope. In further embodiments, a device may be placed or implanted at a particular location in the lung, either before or after a sensor is used to examine the physiological parameters of the delivery site.

According to various embodiments of the present invention, systems and methods for determining the presence of a leak in the lungs as well as the presence of collateral ventilation are provided. The systems and methods may comprise a flow assessment catheter tool that senses and/or measures characteristics of the lungs. Such characteristics can be used to determine the presence of a leak in the lungs as well as the presence of collateral ventilation, for example but without limitation.

In certain embodiments of the present invention, the sensors may be fluid-immersible and capable of minimally invasive evaluation of the physiological parameters of various bodily organs other than the lungs.

In various embodiments of the present invention, the systems and methods for the evaluation of various physiological parameters may include one or more sensors capable of detecting gas exchange, ventilation, perfusion, air flow, collateral path detection, temperature, pH, or various chemical compounds (including volatile organic compounds). Further types of sensors may also be envisaged.

Particular embodiments of the present invention may permit correlation of the information received from the sensors to diagnose various medical conditions. Further embodiments provide for a computer to process the results, and possibly present the results in a human-readable format, including a graphical interface. An operator may then choose an appropriate treatment modality to treat or prevent a medical condition.

### BRIEF DESCRIPTION OF THE DRAWINGS

These and other features, aspects and advantages of the present invention will now be described with reference to several drawings, which drawings are intended to be illustrative and not limiting.

FIG. 1 illustrates an embodiment of a catheter-based tool comprising a distally-positioned sensor extending from the bronchoscope in an airway and a controller in electrical communication with the sensor.

FIG. 2 illustrates an embodiment similar to the embodiment of FIG. 1 but also comprising an occlusion balloon disposed proximate a distal tip of the catheter-based tool.

FIG. 3 is an enlarged view of a distal end of the embodiment of FIG. 2 and employing an oxygen sensor disposed distally of the occlusion balloon.

FIG. 4 illustrates an embodiment of the catheter-based tool in communication with a remote spectrophotometer.

FIG. 5A illustrates an example of a distal tip of the catheter-based tool, wherein the tip comprises wire leads, a portion of the catheter, and a temperature sensor.

FIG. 5B illustrates another example of a distal tip of the catheter-based tool, wherein the tip comprises wire leads, a portion of the catheter, a heating element, and a temperature sensor.

FIG. 5C illustrates a further example of a distal tip of the catheter-based tool, wherein the tip comprises wire leads, a supporting catheter, a heating element, and two temperature sensors.

FIG. 5D shows an example of a graph illustrating a temperature measured at a distal end of the catheter-based tool, wherein the temperature changes with inhalation and exhalation.

FIG. 6A illustrates an example of a procedure for determining implantation sites for treatment devices in a lung. FIG. 6B is representative of a type of output generated by the procedure of FIG. 6A.

FIG. 7 illustrates an example of a procedure for treating air leaks in a lung.

FIG. 8 illustrates an example of a procedure for treating a disease involving an aerobic organism, such as Mycobacterium tuberculosis, in a lung.

FIG. 9 illustrates an example of a procedure for monitoring the treatment of lung tumors.

### DETAILED DESCRIPTION OF THE PREFERRED EMBODIMENT

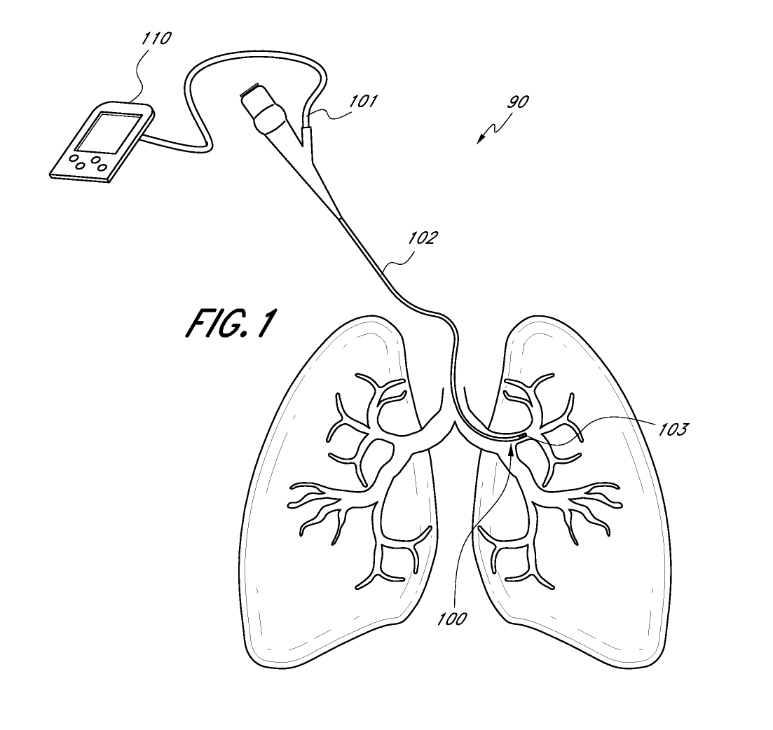

With reference initially to FIG. 1, certain embodiments of a pulmonary diagnostic system 90 that is arranged and configured in accordance with certain features, aspects and advantages of the present invention will be described. The pulmonary diagnostic system 90 advantageously can be used to sense, detect, or otherwise monitor physiological information from within a lung. For example, as will become apparent, some embodiments of the pulmonary diagnostic system 90 can be used to sense air flow or proxies for airflow within, directed to or adjacent to specific regions of the lung (e.g., the lower lobe of the left lung or a portion thereof) or air exchange or air exchange efficiency within specific regions of the lung. Some embodiments of the pulmonary diagnostic system 90 can be used to monitor oxygen concentration within, to or adjacent to specific regions of the lungs. Other embodiments and applications also will be described herein or will become apparent to those of ordinary skill in the art based upon the disclosure herein.

The pulmonary diagnostic systems 90 preferably can be configured for use in measuring any a number of characteristics of the lungs. For example, the pulmonary diagnostic systems 90 can be used to measure temperature changes, air flow rates, differences between inhalation and exhalation air velocities, magnitudes of air flow and/or velocity in a single direction in an airway, concentrations of a specific component or of specific components of a measured fluid (e.g., oxygen concentration) and the like. Advantageously, the measured characteristic can be related to a specific region of the lung instead of being related to the lung as a whole. In other words, the measurement is taken directly within or adjacent to the region of the lung instead of at the mouth or external to the body.

Because certain embodiments of the present invention use a sensor placed in close proximity to a region of interest, those embodiments do not require a gas-subtractive testing method (i.e., drawing air away from the region of interest to a sensor located elsewhere). Because the sensor does not alter the chemistry or physiology in the body region in which the testing is being conducted, the results are more accurate. Advantageously, the sensor disposed within the air passageway is less likely to disrupt the local microenvironment being monitored. For example, drawing air away from an alveolus would affect a concomitant oxygen absorption measurement being conducted at the same time. By reducing the likelihood of disruption of the local microenvironment, physiological measurements conducted are believed to be more accurate of actual conditions than measurements obtain that tamper with the local microenvironment. Accordingly, an advantage of certain embodiments of the present invention that use a sensor placed close to a region of interest is that the sensor may measure gas concentrations without disrupting the local microenvironment adjacent to bronchial regions of interest as would occur with the use of a vacuum or other gas-subtractive methods, thus resulting in more accurate measurements.

At a very high level of description, the pulmonary diagnostic system 90 preferably generally comprises a catheter 101 that comprises one or more sensors 103 disposed at or near a distal end 100 of the catheter 101. The sensor 103 is in communication with a controller 110 such that signals from the sensor 103 can be transmitted to, processed by, and output by the controller 110 or another suitable component. The device can be configured for single patient use or can be configured for resterilization.

With continued reference to FIG. 1, the catheter 101 can have any suitable configuration. Preferably, the catheter has an atraumatic tip. In some configurations, the catheter is designed for insertion into and movement within a working channel of a bronchoscope 102. Thus, the catheter can be compatible for use with flexible bronchoscopy, which allows a doctor to examine an inside of a patient's airway and lungs for abnormalities, such as foreign bodies, bleeding, tumors, or inflammation, for example but without limitation. The flexible bronchoscope can take the form of a long thin tube that contains small clear fibers that transmit light images while the tube bends for navigation of the tortuous bends present in lung air passageways. The flexibility of the instrument allows the instrument to provide readings from very distal locations within an airway. The procedure can be performed easily and safely under local anesthesia. The catheter 101 preferably is compatible with, and axially moveable within, a bronchoscope having a 2.6 mm working channel. In other embodiments, the catheter 101 may be compatible with, and axially moveable within, a bronchoscope having a 2.0 mm working channel. Other configurations also are possible.

In addition, while certain embodiments of the catheter 101 may be designed for use in connection with a bronchoscope, the catheter 101 may be used within other components, catheters or the like or without any other devices. For example, the catheter 101 may be compatible with endoscopes or laparoscopes usable in other environments, including environments where the catheter may be partially or completely immersed in a fluid other than air. These environments may include but are not limited to the gastrointestinal tract, the urogenital system, and other bodily cavities, including those accessed through one or more incisions, such as thoracic organs or the joint spaces. Further, certain features, aspects and advantages of the present invention may be compatible with capsules used for imaging the gastrointestinal tract, such as the EndoCapsule® (Olympus). For example, some of the data gathering characteristics may be useful with the EndoCapsule.

In some embodiments, the catheter 101 may be steerable and flexible, permitting it to be guided to a target location, such as a specific region within a patient's lungs. The catheter 101 preferably is coated or manufactured at least in part from a lubricious material, such as PTFE, FEP, or hydrophilic coatings, for example but without limitation. The lubricious materials preferably are disposed at least on an outer portion of the catheter 101 to facilitate easier movement (such as in the axial direction) of the catheter 101 when inserted into the working channel of the bronchoscope 102. In some embodiments, the catheter 101 may comprise a fixed guidewire that shapes the tip into a shepherd's crook or a similar configuration. Any suitable catheter assembly can be used.

In some embodiments, at least a portion of the catheter 101 can comprise a radiopaque material. Preferably, at least a portion of the catheter 101 proximate to the sensor 103, and/or the sensor 103 itself, can comprise a sufficiently radiopaque material to allow visualization. Such constructions can enable an operator to more easily guide the catheter and/or the associated sensor 103 to a target location using a suitable visualization technique, such as fluoroscopy for example but without limitation.

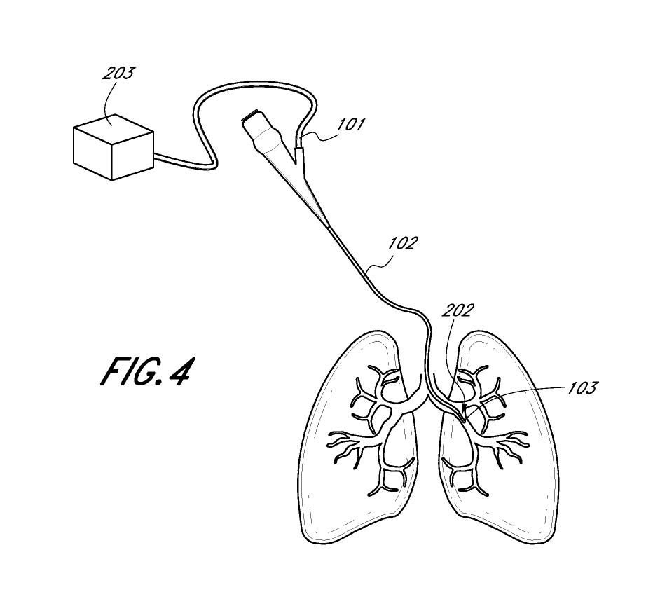

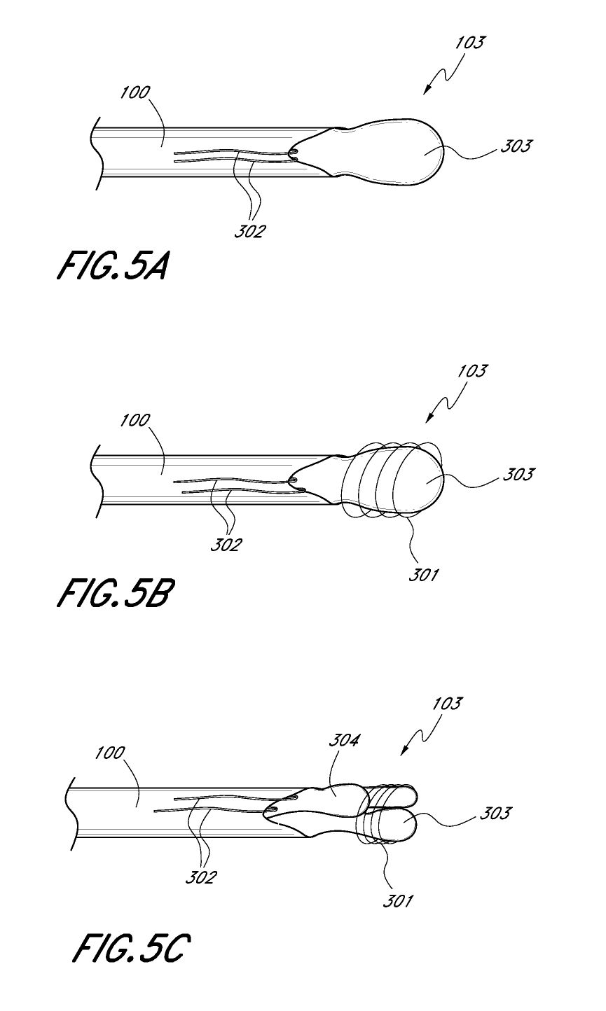

The catheter 101 preferably comprises at least one lumen 106 (see FIG. 3). In some embodiments, the catheter 101 may comprise multiple lumens. The multiple lumens may permit multiple sensors to be introduced or exchanged through the catheter and/or may permit one or more passages through which fluid can be introduced or withdrawn through the catheter, for example. In the embodiment illustrated in FIG. 3, however, the primary lumen accommodates a bundle of wires 205 that extend from the sensor 103 to the proximal end of the catheter 101.

In the embodiment of FIG. 3, the catheter 101 also comprises an occlusion device 105. While the illustrated catheter 101 carries the occlusion device 105, in some embodiments, the occlusion device 105 can be mounted on, can be mounted over or can overlap with at least some portion of the sensor 103.

The occlusion device 105 can comprise a balloon, a one-way valve, or any suitable expanding member such that the occlusion device can be used to isolate particular airways or other body lumens from airflow proximally and/or distally of the occlusion device 105. In the embodiment of FIG. 3, the occlusion device 105 comprises a balloon. The balloon can be inflated and deflated in any suitable manner. In some embodiments, the catheter 101 can comprise at least one lumen dedicated to inflation and deflation of the balloon. Other configurations also are possible.

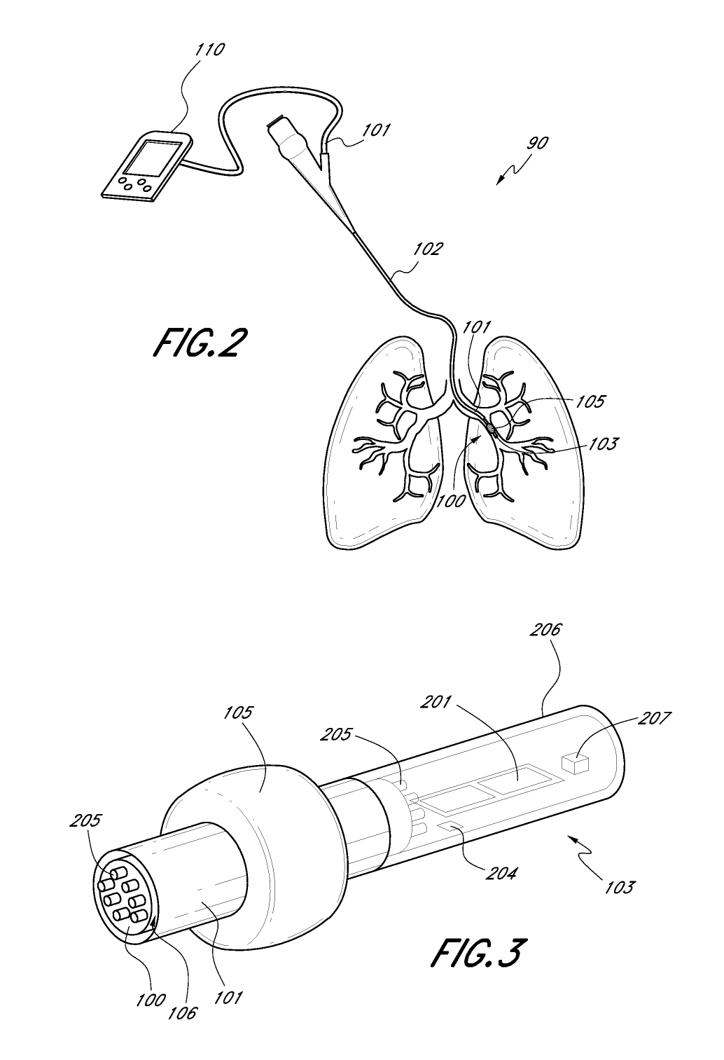

As will be discussed below, the occlusion device 105 may be used during assessment of physiological conditions at selected portions of the lung, such as lung function and/or gas exchange efficiency for example but without limitation. The occlusion device 105 also is useful in the detection of collateral flow, as will be discussed. While the embodiment illustrated in FIG. 2 shows the occlusion device 105 disposed proximally of the sensor 103, it is possible in some embodiments to position the occlusion device 105 distally of the sensor 103 depending upon the data sought with the sensor 103. Moreover, in some embodiments, the occlusion device 105 may be positioned between two or more sensors 103.

In some embodiments, the pulmonary diagnostic system 90 may also be adapted to measure a dimension of the air passageway (e.g., a cross-sectional diameter). For example, the catheter 101 and the occlusion device 105 can be used to measure the cross-sectional diameter or area of the air passageway. Such a configuration can be configured, arranged and used in the manners disclosed in U.S. patent application Ser. No. 10/196,513, filed on Jul. 15, 2002, and U.S. patent application Ser. No. 10/254,392, filed on Sep. 24, 2002, each of which is hereby incorporated by reference herein in its entirety. In some embodiments, a sensor can be provided that can measure airway diameter or cross-section.

As discussed above, the sensor 103 preferably is provided for detecting, measuring and/or monitoring one or more physiological characteristic. Preferably, the sensor 103 is disposed on the distal end of the catheter. More preferably, the sensor 103 provides data with a sufficiently fast response such that a breath-by-breath analysis can be conducted. In one preferred configuration, the sensor can provide information to the operator within five breath cycles. In certain embodiments, the catheter 101 comprises one or more sensors, which may be used to assess various physiological parameters.

The sensor 103 preferably is designed and configured for deployment through a bronchoscope having a 2 mm working channel. In some embodiments, the sensor 103 is designed and configured for deployment through a bronchoscope having a 2.6 mm working channel. The sensor 103 can have a diameter of between about 10 mm and about 0.5 mm. In some embodiments, the diameter is about 0.7 mm. Other sizes, designs and configurations also can be used. Moreover, while the illustrated sensor 103 is described as connected to and supported by the catheter 101, other configurations can feature a sensor 103 that is separate of the catheter and adapted for deployed within the body. In some configurations, the sensor 103 may be mounted to an implantable object such that data can be obtained over an extended period of time. One such object could be a valve or a portion of a valve such as the valve described in U.S. Pat. No. 6,293,951, issued on Sep. 25, 2001, U.S. patent application Ser. No. 09/951,105, filed on Sep. 11, 2001, U.S. patent application Ser. No. 10/081,712, filed on Feb. 21, 2002, United States patent application Ser. No. 10/103,487, filed on Mar. 20, 2002, U.S. patent application Ser. No. 10/124,790, filed on Apr. 16, 2002, U.S. patent application Ser. No. 10/150,547, filed on May 17, 2002, U.S. patent application Ser. No. 10/178,073, filed on Jun. 21, 2002, U.S. patent application Ser. No. 11/204,383, filed on Aug. 15, 2005, U.S. patent application Ser. No. 10/745,401, filed on Dec. 22, 2003, and U.S. patent application Ser. No. 11/585,415, filed on Oct. 24, 2006, each which is hereby incorporated by reference in its entirety and specifically regarding the constructions of the valves and valve components.

In some configurations, the sensor 103 can comprise one or more devices that are capable of measuring temperature. While the sensor 103 measures temperature, the temperature measurement can be correlated to air velocity and, therefore, the sensor 103 can serve as a velocity sensor through detection of a proxy (e.g., temperature changes). Examples of such temperature sensing devices include but are not limited to thermistors, thermocouples, anemometers, electrical thermometers, resistance temperature detectors and the like. In addition, as will be described, some configurations also feature one or more heaters that are positioned close to or generally adjacent to the temperature measure sensors.

Thus, the flow assessment catheter tool can measure air movement in an airway and around valves by measuring temperature or the like. The sensor, which can comprise an anemometer, a thermistor, or other measuring mechanism, may be used to measure energy loss from a heater. Energy loss from the heater can be measured in two ways: 1) by measuring the amount of energy required to maintain a generally constant temperature or 2) by measuring the amount of drop in temperature. Air that passes the heater can heat the sensor. In some embodiments, the sensor comprises an electronic thermometer. Air can transfer heat from the heater to the electronic thermometer. In such configurations, depending on the direction of air flow, air cools the sensor by pushing heat away from the electronic thermometer. In one configuration, air flowing distally heats the sensor while air flowing proximally cools the sensor. In another configuration, air flowing distally cools the sensor while air flowing proximally heats the sensor.

In practice, when air flows over the heater during inhalation or exhalation, the air draws away some portion of the heat from the heater which results in a change in a temperature reading. The change in temperature can then be correlated to certain air flow characteristics. Thus, the velocity, volume or other characteristics of air flow can be detected with the temperature sensor. In some embodiments, the temperature sensors can sense the increase in air temperature caused by the air flowing over the heater rather than the decrease in air temperature over or in the region of the heater.

In some embodiments, the sensor 103 can comprise one or more thermistors. A thermistor is a thermally sensitive resistor that has either a negative or positive resistance/temperature coefficient. The thermistor can be provided in probe form and the thermistor can have a negative resistance/temperature coefficient. In some embodiments, however, the thermistor is provided as a glass bead, disc, chip or any other suitable form and/or the thermistor can have a positive resistance/temperature coefficient.

As discussed above, the sensor 103 also can comprise a heater such that the thermistor is positioned on one or both sides of the heater. In other words, one or more thermistor can be positioned on each side of the heater (i.e., a thermistor can be mounted on a proximal side of the heater to specifically sense a velocity of air flow, or an amount of temperature change caused by air flow, in a distal direction and a thermistor can be mounted on a distal side of the heater to specifically sense a velocity of air flow, or an amount of temperature change caused by air flow, in a proximal direction). The use of two or more thermistors straddling one or more heaters can allow for differentiation between distal and proximal flow. Thus, air flow in the inhalation direction and air flow in the exhalation direction both could be sensed.

The heater can have any suitable configuration. Some embodiments comprise a copper heater with four turns or coils that are tightly packed around the thermistor. Some embodiments comprise a nichrome heater with four turns that are tightly packed around the thermistor. Generally speaking, when the turns are tightly packed, there is little or no air space between the thermistor and the copper or nichrome. Being tightly packed prevents or at least greatly reduces the likelihood of fast cooling of the heater. Some embodiments have at least four coils and a larger diameter wire. In some embodiments, the coils comprise a 37 AWG wire, which has a diameter of about 0.0045 inch.

In some embodiments comprising multiple (e.g., two) thermistors, the proximal thermistor and the distal thermistor respond similarly. It is typical for the thermistors to be 180° out of phase. In some embodiments, the heater can be made larger in diameter and can be generally oval-shaped. A greater surface area of the heater can be placed in front of the proximal thermistor. In some configurations, the distal thermistor may heat too much and the air temperature may not change much. In this instance, the coils can be loosened so that they do not adhere directly to the distal thermistor. In another embodiment, the coils can be placed closer to the proximal thermistor, which heats and cools with every half breath. When the coils are placed farther away from the proximal thermistor, the proximal thermistor reads a slightly higher temperature on expiration.

When two or more thermistors are used to measure the magnitude of the air velocity, the actual direction of flow or velocity is not directly measured. Instead, the direction of flow can be “gated” with respiratory rhythm. In some embodiments, the temperature profile of inspiratory and expiratory velocity may be differentiated by mating the temperature data with data from a “gate.” In some embodiments, a thermistor can be placed in the main airway. In other embodiments, a flow switch or “gate” can placed in the main airway in series with the trachea tube. In other embodiments, a flow meter or flow direction sensor can placed in the trachea. A valve can also be placed in the air passageway. The valve may be a one-way ball valve, a flap valve or any other suitable valve. The valve may assist in gathering the flow direction or “gate.”

In some embodiments, the sensor 103 comprises an anemometer. The anemometer measures velocity. The anemometer can act as a mass flow meter. Air is forced around the anemometer (by the lungs) to convectively transfer heat away from the sensor 103. In most embodiments, the anemometer may be a hot-wire anemometer. Hot wire anemometers use a very fine wire heated to a temperature above the ambient temperature. The wire diameter may be on the order of several micrometers (e.g., a filament). The filament may be comprised of nickel-chromium (i.e., nichrome) wire. In some applications, the filament may be comprised of a material with a high resistance. The higher resistance is desired so that lower current can be used. Air flowing past the wire has a cooling effect on the wire. Because the electrical resistance of most metals is dependent upon the temperature of the metal, a relationship can be obtained between the resistance of the wire and the air velocity. In some embodiments, the hot-wire comprises tungsten. Hot-wire anemometers, while delicate, have a high frequency-response and fine spatial resolution compared to other measurement methods and as such are preferred for the detailed study of turbulent flows or any flow in which rapid velocity fluctuations are of interest.

In other embodiments, the sensor comprises at least one thermocouple. A thermocouple is a temperature sensor that can be used as a means to convert thermal potential difference into electric potential difference. Thermocouples are inexpensive and interchangeable, have standard connectors, and can measure a wide range of temperatures. Thermocouples are smaller than thermistors. Thermistors may have difficulty getting sufficient air flow, unlike thermocouples that are small enough in size to receive enough air flow to make more accurate measurements. Thermocouples may be electrically noisier than thermistors. Further, thermocouples may be more difficult to fixture to suitable computer systems or control systems.

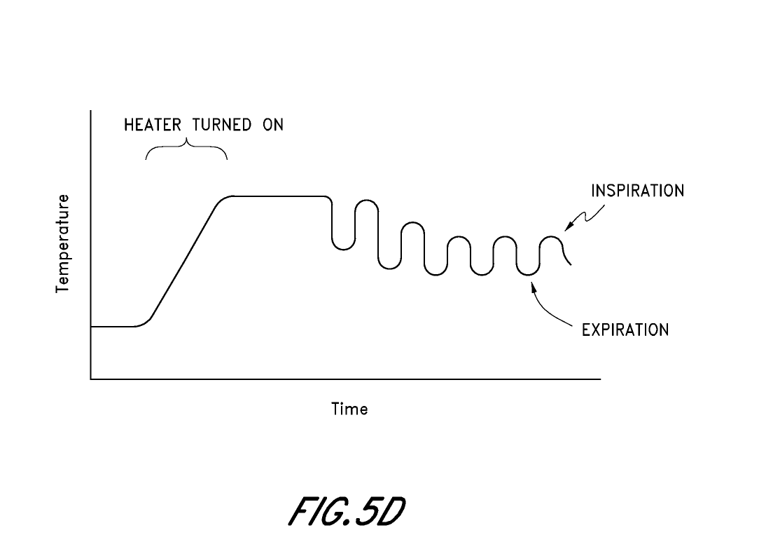

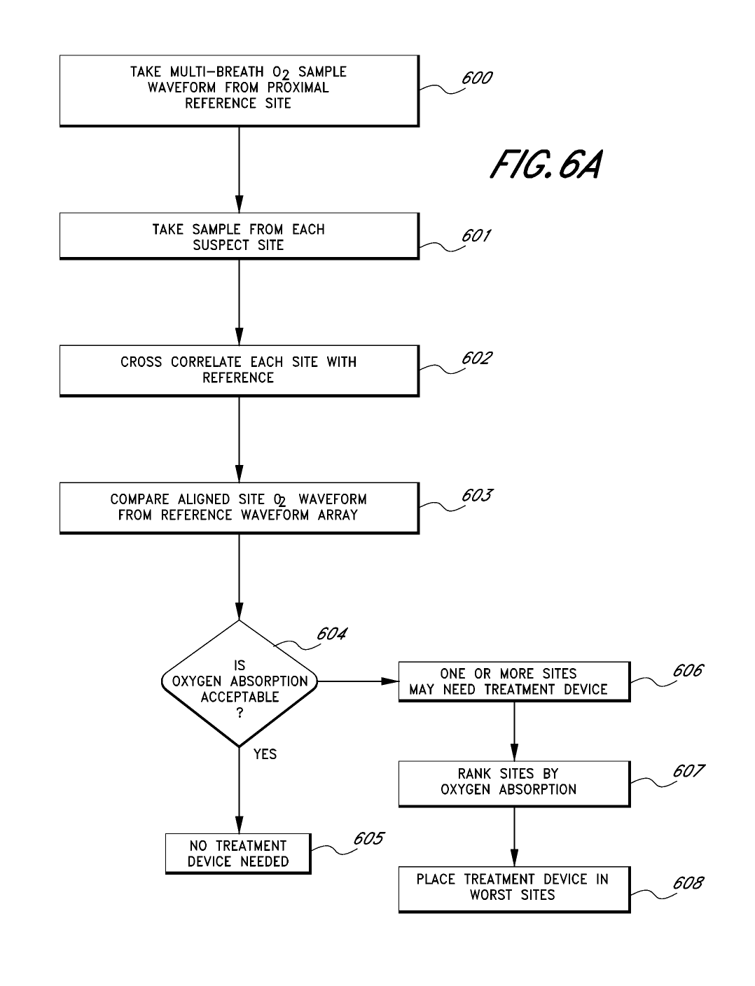

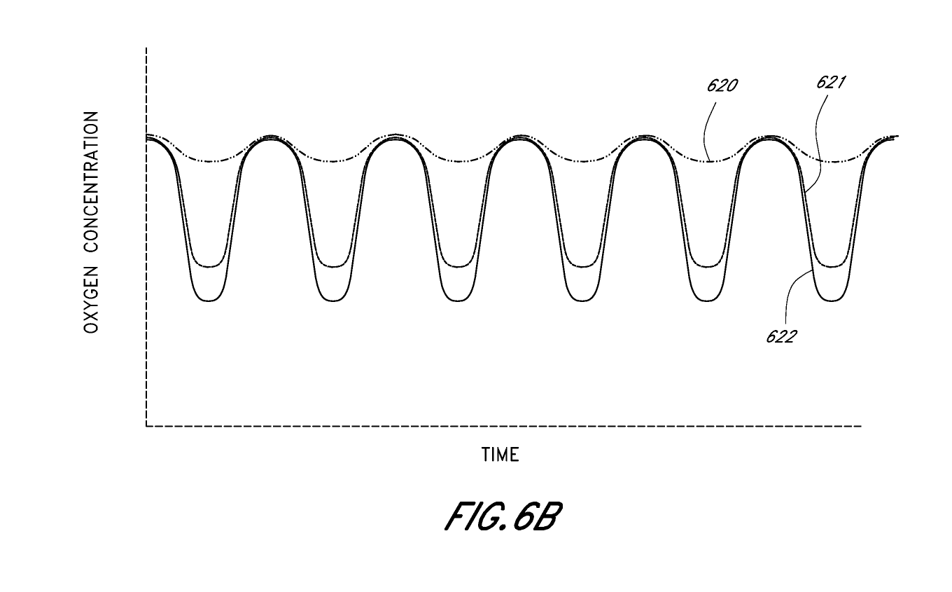

With reference now to FIG. 5A, the catheter 101 may comprise a distal end 100 with a sensor 103 mounted thereupon. The sensor 103 may comprise a temperature sensor 303 disposed at its distal end with wire leads 302. As described above, the temperature sensor 303 may be any suitable configuration and can comprise, for example, a thermistor, a thermocouple, a resistor able to measure temperature changes, or any other type of sensor able to measure temperature. In some configurations, the temperature sensor 303 may be used as a mass flow meter. Air may be forced or directed around the sensor 303 to convectively transfer heat to or away from the temperature sensor 303.

In some embodiments, the catheter 101 comprises at least one heater, as illustrated in FIG. 5B. The distal end 100 of the catheter 101 comprises a heating element 301. In some embodiments, the temperature sensor 303 may be mounted on the distal side of the heating element 301. A small amount of electricity may power the heating element 301. In certain embodiments, the heating element 301 comprises a conductor with high resistance, for example nichrome. In certain embodiments, the conductor may be looped around the temperature sensor 303. The sensor can also be self-heating and not require a separate heater or electrical connections. In certain embodiments, the heating element 301 can be replaced with a cooling element, for example a Peltier chiller. When the temperature sensor 303 is a thermistor, the resistance in the thermistor can be sensed at the same time as the electricity sent to the thermistor is measured. The amount of current supplied can be proportional to the resistance in the thermistor. The resistance in the thermistor is proportional to the temperature, and the temperature is proportional to the air speed. In most configurations, the catheter tool preferably operates on the thermodynamic principle of forced convection heat transfer.

In other embodiments, for example as depicted in FIG. 5C, a second temperature sensor 304 may be mounted on the proximal side of the heater to specifically sense the velocity of air flowing in a distal direction. Meanwhile, the first temperature sensor 303 is able to sense the velocity of air flowing in a proximal direction. The presence of two temperature sensors would allow for differentiation between distal and proximal fluid flow. In one configuration, inspiration would warm the first temperature sensor 303 while expiration would warm the second temperature sensor 304.

FIG. 5D illustrates an example of a graph of the temperature response over a certain time period of an embodiment equipped with a temperature sensor and a heater. The sharp upward slope at the beginning of the time period measured represents the temperature rising as the heater is turned on. After reaching steady-state, the heater may be inserted into a patient's airway. The temperature will then rise and fall based on when the patient breathes in or out. Using these collected temperature measurements, various calculations are possible, such as determining air flow velocity in a lung passageway.

In certain embodiments, the catheter 101 can be provided with a sensor to detect one or more gases, gas components, fluid components or other substances. For example, there may be a sensor to detect oxygen or carbon dioxide concentration. In the embodiment illustrated in FIG. 3, the sensor 103 may comprise an oxygen sensor 201. An oxygen detector 201 may be provided, in addition to a processor 207. Power and data may be transmitted via wire leads 205. Other configurations also are possible. The oxygen sensor 201 may also comprise a temperature sensor 204. The oxygen sensor preferably comprises a cover 206 to protect the internal components of the oxygen detector 201.

Certain embodiments of the present invention also provide for similar sensors able to detect different types of gases, for example carbon dioxide. With regards to oxygen sensors, various commercially-available oxygen sensors may be employed, such as the SM100-02 sensor (SMSI, Germantown, Md.) which functions based on the oxygen quenching of a fluorescent molecule. The fluorescence quench oxygen sensor can be positioned at the distal end of the catheter and can generate electrical signals as a function of an instantaneous oxygen content of the respiratory gases. A computation unit can receive the output signals from the sensor and from a flow sensor (e.g., temperature sensors) to calculate oxygen concentration and related parameters.

In FIG. 4, an embodiment is shown where the sensor 103 functions spectrophotometrically; in such cases, a transmission component such as a fiber optic cable 202 may be used to relay spectral information from the sensor 103 to a remote spectrophotometer 203 able to detect the various chemical entities present near the sensor.

In further embodiments, the device comprises a sensor used to detect and measure the temperature of the local microenvironment of the lung to diagnose various medical conditions. In some embodiments, a sensor can be provided with a hydrogen ion sensor (pH sensor). Such sensors may be useful in the diagnosis and detection of tissue inflammation, cancer, or bacterial and viral diseases. Further, a sensor may be provided to detect volatile organic compounds or other biomarkers indicative of disease states, and the system may be configured to detect and measure such compounds as predictors of various disease states such as cancer. In some embodiments, the sensor measures air or gas pressure in the lung.

The catheter 101 may relay data to a control system, for example a hand held device. The control system receives data from the catheter and/or sensor and processes the data. In some embodiments, the catheter and/or sensor can connect directly into an analog-to-digital (“A/D”) converter that converts continuous signals to discrete digital numbers. In other embodiments, the catheter and/or sensor can connect directly into a compact flash A/D converter for the control system. In some embodiments, the catheter and/or sensor can connect wirelessly to the control system. In some embodiments, the sensor can receive power from the control system. The digital output may use different coding schemes, such as binary and two's complement binary. The code may be written in Labview or any other suitable code to access the A/D code, process the data and send signals back to the catheter, if desired.

The device may measure the relative difference between the temperatures of inhaled air and exhaled air. As discussed above, the difference in temperature then can be used to compute the difference in air velocity between inhaled air and exhaled air. The device may also measure other physiological parameters, such as gas concentration (including oxygen concentration), temperature, and pH. While the catheter may be operated by one individual, the control system may be operated by a separate individual. In some configurations, both the catheter and the control system can be operated by a single individual.

In some embodiments, the device may generate or cause feedback such as audible output or tactile output, for example. In some embodiments, the device may measure physiological changes, including temperature and/or temperature changes in an air passageway, and then generate feedback related to parameters such as airflow velocity, oxygen concentration, or temperature. For instance, the slope of the temperature, related to velocity or flow, as the temperature drops or raises can be converted to one of the three audible sounds: a) amplitude of sound waves, b) frequency of sound waves, and c) number of sounds beeps. The sounds output from the device could be correlated to any other physiological parameter measured by the sensor. In some embodiments, the computer, controller or device comprises output speakers. The audible sound signals can be directed to the output speakers.

In some embodiments, the device comprises a trigger. The operator of the device can push the trigger, which would send a signal to the control system. By pushing the trigger, the operator could indicate to the system to start or stop taking data.

In some embodiments, the sensor or a portion of the catheter proximate to the sensor may be provided with or associated with location-tracking components, such that a device may gather location data from the sensor or a portion of the catheter proximate to the sensor to create a map or other representation of respiratory passages. In some embodiments, mapping may be effectuated by reference to the distance that a catheter containing the sensor has traveled, or by electronic tracking means located on the distal end of the catheter or the sensor. In some embodiments, a device may correlate such location data with other physiological data sent from the sensor, which facilitates the creation of a map or other representation of respiratory passages to be correlated with the physiological data received from the sensor. For example, a map of ventilation efficiency, airflow, oxygen concentration, or carbon dioxide concentration in a patient's lungs may be created.

In some embodiments, the data collected from the sensors may be used to model or simulate the function of various bodily organs. For example, information gathered from one or more oxygen sensors may be used to calculate oxygen exchange in various portions of the lung. By virtue of the small size of the device, sensors may be able to detect and correlate gas concentrations in discrete areas of the lung, unlike traditional gas exchange methods which are only able to measure gas concentration at the mouth or throat.

Certain embodiments provide for aggregation of the data collected from the sensors. Lung function may then be mapped to discrete zones of the bronchial anatomy. For example, this mapping may be used in emphysemateous patients to determine an optimal site for treatment, which may include the determination of implantation sites for an occluding device or one-way valve. This mapping may be done with a graphical representation of a patient's lungs, thereby depicting regions of the lung with better or worse physiological parameters. Such parameters may include, but are not limited to, mapping oxygen exchange and air flow (including measurements of inspiratory and expiratory volume). In certain embodiments, these parameters may be compared to reference measurements, for example from standardized data sources, or segments of the own patient's lungs that are known to be healthy.

Information obtained from sensors, either with or without mapping discrete areas of the lung to a general lung map, may be used concordantly to determine bronchi that would benefit most from the implantation of a medical device, such as a one-way valve or a bronchial occluding device. For example, in some embodiments, the sensor can detect the sections of the lung that are not functioning properly. This may be accomplished, for example, by using an oxygen sensor, as described above, to determine oxygen extraction from a portion of the lung. This may be useful in the diagnosis of emphysema or COPD.

In some embodiments, the sensor measures air flow in the lungs as well as air flow out of the lungs. If the lung tissue is diseased or necrotic and cannot exchange much air, the sensor can be used to identify the segments of the lung containing such tissue. The segments of the lung containing diseased tissue have the least amount of air movement and air flow into the lung during inspiration or out of the lung during exhalation. Measuring air flow is also a way to detect the existence of asthma. A segment of a lung inflicted with asthma may have a higher velocity of air.

In certain embodiments, the device 90 may be used to direct placement of a medical device. A valve, such as the valve disclosed in U.S. Pat. No. 6,293,951 for example but without limitation, which is hereby incorporated by reference in its entirety, can be placed in the lung with the guidance of the sensor. Placement of a valve in an air passageway may be based on Computed Axial Tomography (“CAT” or “CT”) scans or other suitable medical imaging system output. In addition to medical imaging, valve placement in the air passageway can be based on data sensed by the sensor. The sensor facilitates a more efficient identification of a desired location for placement of the valve. Further, patients receive optimal acute treatment. Additionally, the sensor can be used to verify sealing of the air passageway with the valve by checking for air flow, as will be described below. Thus, there is a reduced likelihood of requiring follow-up implants. In fact, the sensor can be used anytime before or after valve placement in-vivo.

If the one-way valve leaks (i.e., the valve permits air to flow in a distal direction when the valve is designed to prevent or greatly restrict flow in the distal direction), the leaks can be identified by sensing air flow. For example, when temperature is sensed by a dual thermistor construction discussed above and when the temperature is used as an indicator of air flow, a larger temperature differential on a distal temperature sensor 303 and a smaller temperature differential on a proximal temperature sensor 304 would be indicative of an air leak. This is because, during inhalation, the inspiration can cool the air around the proximal thermistor and expiration (occurring when air is released by the one-way valve) can cool the distal thermistor. In some embodiments, the inspiration results in heating of the air proximate the distal thermistor while the expiration results in heating of the air proximate the proximal thermistor. When a one-way valve is venting air (i.e., the valve permits a large amount of air flow in a proximal direction, for example due to a hole in the valve) the opposite effect would take place: a venting valve would result in a large temperature differential on the proximal sensor 304 and a small temperature differential on the distal sensor 303 compared to a valve that does not vent. A valve that was both venting and leaking would result in both the temperature sensors 303 and 304 having similar temperature differentials.

FIG. 6A is an exemplary flowchart for a procedure that may be used to determine implantation sites for lung treatment devices, which may include one-way valves. The procedure preferably makes use of a sensor that can sense a change in oxygen concentration or a sensor that can sense changes in other components of the air. In some embodiments, the air flow rate or volume also can be used.

In procedure illustrated in FIG. 6A, an operator may obtain an oxygen sample waveform, which represents a change of oxygen concentration over time in an airway, from a reference site. See 600. Preferably, the reference site can be located in one of the larger bronchial tracts (e.g., the left or right main bronchus) or from the mouth. This oxygen sample is preferably taken over a longer period of time (e.g., over several breaths) and can provide a baseline sample against which the remaining samples can be compared.

Next, an oxygen sample waveform can be taken from sites located deeper in the lungs (e.g., along several smaller bronchioles). See 601. An operator may choose to take samples only from sites suspected of having a respiratory abnormality, such as emphysema for example but without limitation.

The oxygen sample waveforms from these distal sites are then correlated and compared to the reference site. See 602, 603. The sampled sites may then be organized according to the oxygen waveforms. Based at least in part upon the waveforms, an evaluation can be made regarding the level of oxygen exchange or absorption. See 604. In other words, an operator may determine whether a particular test site has an oxygen exchange (e.g., a lower level of exhaled oxygen concentration) higher or equal to the reference site or whether a particular test site has a lower oxygen exchange (e.g., a higher level of exhaled oxygen concentration). Stated another way, an operator may evaluate whether the particular test site adds or detracts from the overall level of oxygen extraction by the lung. If the level of oxygen absorption or exchange is satisfactory, then no treatment, such as the insertion of valves, may be indicated. See 605.

If sites are identified in which poor oxygen absorption is evidenced by a low or nonexistent change in oxygen concentration over time, then treatment such as installation of one-way valves or other bronchial occluding devices can be recommended. See 606. The sites from which samples were obtained can be ranked and identified as possible candidates for treatment. See 607. Through a progression of installations, the sites evidencing the least amount of oxygen absorption or exchange can be treated first. See 608. In some configurations, a predetermined cut-off value for the change of oxygen concentration over time may be used that is indicative of a likely disease condition, such as emphysema for example but without limitation, and such a cut-off value used to determine sites as possible candidates for treatment. In some such embodiments, a catheter carrying a sensor used as described above advantageously also may carry a treatment device such that a device may be implanted immediately following site evaluation by the sensor where the evaluation indicates an oxygen exchange level lower than a set reference value that indicates treatment is desired.

With reference to FIG. 6B, a set of waveforms representing examples of oxygen concentration valves over time are presented. These waveforms are predicted and are for illustration purposes only and are not necessarily reflective of actual data that may be observed during the procedure described above. An oxygen waveform 620 from a first sample site shows far poorer oxygen absorption than an oxygen waveform 622 from the reference site, indicating that such a site may benefit from treatment. Oxygen waveforms taken from other sites that show an oxygen waveform similar to the reference site oxygen waveform 622 indicate that treatment of such sites may not be needed. After a site has been treated, for example with a one-way valve, the efficacy of the treatment may also be verified. For example, oxygen waveform 621, taken from the first sample site after treatment, shows an improvement in oxygen absorption compared to oxygen waveform 620 taken from the same sample site before treatment.

In some configurations, a site that has a treatment device implanted therein may be tested subsequent to implantation. For example, in a larger lung passageway feeding several smaller lung passageways, wherein at least one smaller lung passageway may be occluded with a treatment device, airflow or oxygen waveform measurements may be taken before and after occlusion with a treatment device. In some configurations, the airflow or oxygen waveform measurements taken proximate the treatment device can be compared to a reference measurement. The data taken prior to implantation and the data taken following implantation can be taken to see whether improved oxygen exchange has resulted.

In some embodiments, a physician can determine whether collateral ventilation exists within a region of the lung by assessing the data sensed or measured by the sensor and relayed to the control system. More preferably, the sensor can be used to detect the presence or occurrence of collateral ventilation. Because some embodiments of the sensor can be used to detect gases (e.g., helium, oxygen, and carbon dioxide), gas concentrations or changes in gas concentration, the sensor may be connected to additional equipment capable of performing analyses on output from the sensor to determine the presence or levels or changes in levels of such constituents.

In some embodiments, an air way feeding a particular lung portion can be occluded such that the air flow to that lung portion is stopped. A substance, such as a tracer gas for example but without limitation, can be injected into the isolated lung portion while the sensor is used to sense the presence of the tracer gas in another lung portion or while the sensor is used to monitor the concentration of the tracer gas. The concentration of the substance detected either in the isolated lung portion or in another lung portion may be proportional to the amount of collateral ventilation between the two segments.

In some embodiments, if collateral ventilation exists, output from the sensor may indicate that a particular lung segment has a higher velocity of air flow in one direction (i.e., during inhalation or during exhalation). For example, if collaterals are feeding a segment that is only venting air out, then the sensor would sense a greater velocity of air flow out from that segment during exhalation. As a corollary, if the sensor detects more airflow in an air passageway moving in a distal direction during inhalation than in a proximal direction during exhalation, the air passageway can then be treated to reduce collateral ventilation to other lung segments, portions or lobes. Treatment may include blocking the air passageway or some portion of the air passageways that connect to that air passageway.

As discussed above, the device 90 can compute measurements based on data relayed from the sensor. For example, the device can compute an average temperature in the lungs. In some configurations, if the lung has a leak, the sensed temperature generally is lower than normal due to the movement of air passed the sensor in a distal direction, which can then indicate the extent of the leak in the lung. In such configurations, if the sensed temperature is substantially lower or starkly lower than average, then the leak in the lung is usually larger in size. If the sensed temperature is only slightly lower than average, then the leak in the lung is small in size. Once the leak is detected, the physician can then place a valve into the airway to block the leak. In some configurations, as the sensor is moved within the lung, the proximity to the leak can be detected through fluctuations in temperature. In other words, the temperature changes may appear within the portion of the lung feeding the leak.

A leak in the lung tissue may cause more inspiratory air flow or velocity to enter the lung and less expiratory air flow or velocity to exit the lung. In the presence of a leak, the sensor can be used to discern that a magnitude of inspiratory air velocity is greater than expiratory air velocity. In some configurations, measuring flow may require the use of an airway diameter measuring catheter in order to achieve a more accurate reading. In some embodiments, in order to determine if a leak is present in the lungs, the sensor may detect or measure an amount of bubbles and surfactants present in the lungs. In some embodiments, the sensor detects or measures sound using lung or chest auscultation and is capable of listening to the internal sounds of the respiratory system. The sensor then relays the information to a processing unit whereby the system determines if a leak is present. For example the airflow of a lung with a leak may produce sounds that are a different pitch or a different length of time than the sounds of a normal lung.

In some embodiments, the sensor measures or senses the difference between the inhaled and exhaled air velocity. The sensed data or measurement can be used to determine if a leak exists in a portion of the lung distal to the sensor. Air flows distally down the airway toward any existing leak. If the lung has a leak, less air will flow proximally away from the leak. In one embodiment, air flowing distally heats the sensor. The sensor measures the change in temperature and, in some embodiments, the rate of change in temperature also can be calculated. In one configuration, an electronic thermometer, which can be the sensor in such a configuration, is heated from the increase in distal airflow. In another configuration, the electronic thermometer is cooled by the increase in distal airflow. In some configurations, air flowing proximally cools the sensor. The cooler temperature drops slower as there is less air flowing proximally. Thus, the sensor can be used in smaller or progressively smaller airways until the leaky airway is pinpointed.

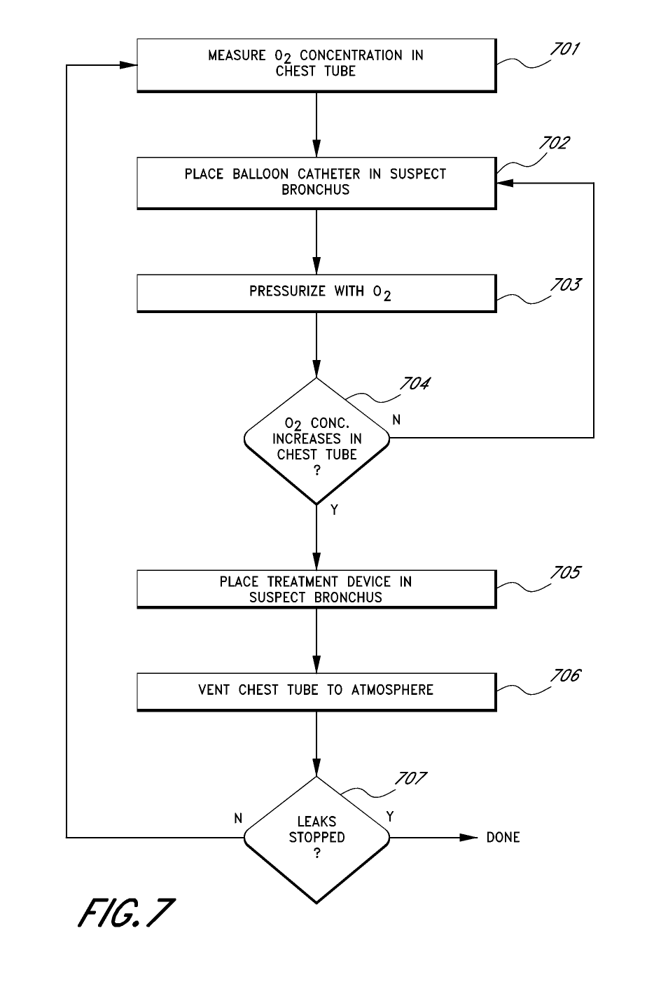

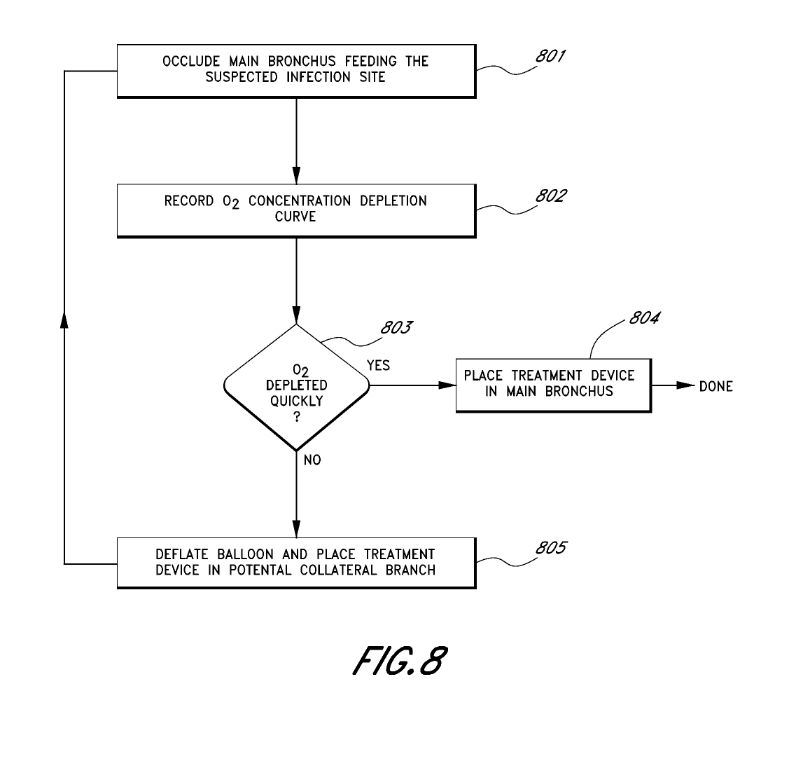

With reference to FIG. 7, air leaks in the lung, such as those occurring incidental to a pneumothorax, can be identified in another manner. In the illustrated method, a chest tube may be provided to drain air or fluid from the pulmonary intrapleural space. In some embodiments, a Heimlich valve or suction can be applied to the chest. A sensor, for example an oxygen sensor, may be inserted into a chest tube. A reference oxygen level in the chest tube may then be determined. See 701. Selected lung passageways may then be isolated (with, e.g., an occluding balloon, a one-way valve or other suitable obstructing member) and the isolated portion of the lung can be pressurized oxygen, preferably substantially pure oxygen (or other gases, including inert gases). See 702, 703. The sensor then can be used to monitor the gas concentration in the chest tube. See 704. If the gas concentration does not increase within the chest tube, then the pressurized is not passing through the leak and into the intrapleural space. Thus, another passage should be occluded, pressurized and checked. The process of selecting and occluding air passages can be continued until the air passage feeding the leak is identified by an increase in the concentration of oxygen detected in the chest tube and a treatment device, such as a one-way valve or the like, can be inserted in the passage feeding the leak. See 705. With the passage feeding the leak occluded, the chest tube can be vented to the atmosphere (see 706) and the success of the treatment can be evaluated (see 707). An advantage of this technique is that the pressurization of the lung portion containing the leak helps maintain the leak in an open position for identification and treatment and only allows the leak to close following identification and treatment.

In another exemplary procedure, lung infections by aerobic organisms such as Mycobacterium tuberculosis for example but without limitation may be diagnosed and treated using the systems disclosed herein. Because aerobic organisms require oxygen, their presence may be determined by comparing the oxygen absorption of a particular lung segment and comparing it to a reference lung segment. In certain embodiments, detection of lung infections not limited to aerobic organisms is possible through the use of a sensor able to detect temperature, pH, volatile organic compounds, or other biomarkers indicative of infection. For example, anaerobic organisms causing a lung abscess would not typically consume oxygen, such that detection via oxygen absorption may be difficult if not impossible. Detection and diagnosis of infection may be effectuated, for example, by comparing the biomarkers with those of a section of the lung known to be healthy. Particularly in the case of volatile organic compounds, detection and diagnosis may also be performed with reference to known quantities or concentrations of compounds being present as being indicative of infection. A lung portion determined to be affected by an infection may then be treated by occlusion. The occlusion can be used to prevent oxygenated air from reaching in infected region. Advantageously, the treatment may be targeted only to the area of the lung known to be affected or believed to require prophylactic treatment.

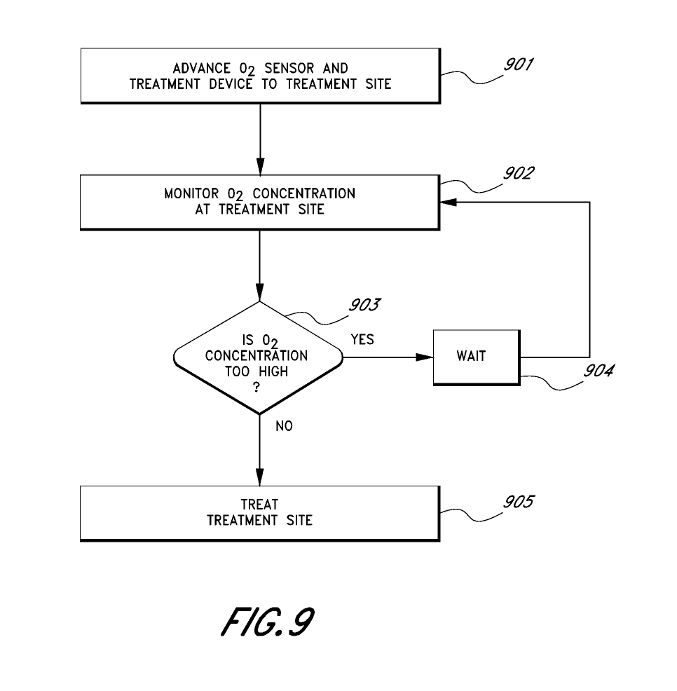

Referring to FIG. 8, following occlusion of air passages that surround a region of the lung to be treated, a catheter with an occlusion device, such as a balloon, may be advanced to a bronchus feeding a portion of the lung believed to be infected by an aerobic organism. The main lung passageway may then be occluded with the occlusion device. See 801. With the lung passageway occluded and a portion of the lung isolated, the lung may be ventilated with 100% oxygen, for example without limitation. With the sensor positioned distally of the occlusion device, an oxygen waveform showing oxygen depletion over time can be obtained. See 802. If the oxygen is depleted at a rate that is faster than a reference oxygen depletion rate (obtained, for example, from a healthy portion of a lung), a permanent or semi-permanent occlusion device or valve may be placed at the site occluded during the oxygen measurements. See 804, 805. Moreover, if the oxygen concentration approaches zero in the target portion of the lung, then all collateral passages are likely occluded. If, however, the oxygen is not depleted at a relatively fast rate, there is a possibility that the site is being fed oxygen from a collateral passageway, or that the site being monitored itself may be a collateral passageway. In such a case, treatment may include repeating the procedure on another passageway suspected to be feeding the diseased portion of the lung and/or occluding another collateral passageway that may be feeding the first site measured. See 805. The collateral air flows can be identified in any suitable manners, including but not limited to those set forth herein. With the collateral air passages occluded, the test can be started again.

As explained above, the sensor can measure a velocity of fluid flow. Measuring the velocity of fluid can be used to detect a narrowing in the bronchial airways. Narrowing of the bronchial airways may be caused by a tumor or by asthma, for example but without limitation. The catheter may be threaded down an airway beyond a target location identified visually or by any suitable type of medical imaging. Typically the airway decrease in size in a distal direction. Thus, during retraction, the velocity of the air flow would be expected to decrease. When the catheter is retracted, if the catheter senses an increase in velocity, a narrowing of the airway may be present. Accordingly, possible tumor locations can be identified by identifying flow restrictions within the airways.

With reference to FIG. 9, certain embodiments of the present invention provide for use of oxygen sensors in the course of the treatment of dysfunctional lung tissue, including lung tumors. Lung tumors may be detected using the various embodiments disclosed herein, although they may also be detected through other means known in the art. In the case of lung tumors, certain tumors may partially or completely occlude lung passageways, and treatment may consist of laser ablation or other treatment devices that carry a risk of combustion. Because some patients may be breathing an oxygen-enriched atmosphere, laser ablation and similar treatments sometimes carry the risk of combustion in the lungs, a decidedly undesirable consequence.

Therefore, in some embodiments, the oxygen sensor is positioned in proximity to an area to be treated. See 901. The oxygen concentration may then be measured prior to and during treatment. See 902. If the oxygen concentration is too high, such that a risk of combustion exists, an operator may take steps to reduce the local oxygen concentration (for example by reducing the oxygen concentration of the oxygen-enriched atmosphere breathed by the patient, or at least partially occluding a lung passageway to be treated), and wait for the oxygen concentration to decrease. See 903, 904. In some embodiments, the system can be integrated with the laser treatment device such that the laser is automatically shut off when the oxygen concentration level exceeds a predetermined threshold. If the oxygen concentration in the vicinity of the treatment site is low enough that there is no longer a significant combustion risk, an operator may choose to start, continue or maintain the treatment procedure. See 905.

As described above, the system advantageously allows direct monitoring within the lung. While the systems described above feature one or more sensor at a distal end, it is possible for one or more sensors to be positioned at various locations along the length of the catheter such that measurements can be taken simultaneously or nearly simultaneously or at differing intervals and at different locations along an air way. For example, a proximal sensor can generate a baseline sample or reference while a distal sensor could provide a site-based sample simultaneously. In addition, in some embodiments, one or more of the sensors may be detachable and capable of wireless operation.

Accordingly, the systems and methods above result in a number of other advantageous methods. In some configurations, because the performance of individual lungs and/or lung segments can be investigated, lung implant procedures can be improved by allowing the better of two lungs to be identified in a donor or recipient. Moreover, mechanical ventilation can be improved by focusing upon the ability of the lungs to participate in oxygen absorption or exchange, for example but without limitation. Furthermore, information can be obtained for evaluation of the lower lobes of lungs, which generally are not used for tidal volume breathing. In some applications, a comprehensive analysis can be conducted of the lungs to determine overall lung condition and health. Such an analysis can provide useful data before proceeding with any other types of treatments. In some applications, the system can function as a diagnostic tool for identification and/or treatment of pulmonary embolism and the system can be used to monitor and examine lung improvement during and/or after treatment. The system can detect a shift in lung volume as a technique for determining where an embolism is located. The system can be used for reviewing, analyzing and/or conducting a VQ scan or lung ventilation/perfusion scan. In some embodiments, the system can be configured to use a multilumen balloon (and/or balloon catheter) that allows a user to pump in and out gases while monitoring gas concentrations. In some embodiments, the pumping and sensing can be performed proximally relative to the balloon. Moreover, in some configurations, a respiratory exchange ratio or respiratory quotient could be mapped by occluding a portion of a lung and independently ventilating the occluded portion of the lung while monitoring the gas exchange within that portion of the lung such that inefficient lung segments can be identified for treatment.

One or more of the embodiments and/or methods described above provide a pulmonary diagnostic system for measuring one or more of a number of parameters related to pulmonary function, which parameters may be used in diagnosis, treatment and monitoring of a lung of a patient. The terms “patient” and “subject” as used herein may refer to mammals, including humans and animals, such as primates, dogs, cats, sheep, cattle, goats, pigs, horses, rats, mice, rabbits, guinea pigs, and the like, for example but without limitation. The terms “patient” and “subject” may be used interchangeably. Moreover, as used herein, the term “proximal” and the term “distal” each shall have its ordinary meaning and, specifically, “proximal” means toward a mouth or in a direction indicated as leading outward from a body and “distal” means toward a lung or in a direction indicated as leading further inward in a body.

Although the inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. For example, while certain features, aspects and advantages of embodiments of the present invention have been disclosed in the context of usage within a lung or its associated passageways, some features, aspects and advantages of the embodiments of the present invention may find utility with other body lumens or cavities. For example, the system can be configured for ischemic bowel applications or can be configured for gastro esophageal reflux disease applications. In some embodiments, the system can be configured for sleep apnea applications. For example, the system can be configured to be implanted and/or to help implant a device that can stimulate a muscle response in response to oxygen depletion that may occur during suspended breathing. In addition, while a number of variations of the inventions have been shown and described in detail, other modifications, which are within the scope of the inventions, will be readily apparent to those of skill in the art based upon this disclosure. It is also contemplated that various combinations or subcombinations of the specific features and aspects of the embodiments may be made and still fall within one or more of the inventions. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above.

What is claimed is:

1. A device for assessing physiological parameters of a lung, comprising:
a catheter comprising a proximal end and a distal end, the catheter being adapted to fit into a bronchoscope; and
at least one sensor disposed at the distal end of the catheter, the sensor capable of detecting one or more physiological parameters of a lung.

2. The device of claim 1, wherein the sensor comprises an oxygen sensor

3. The device of claim 1, wherein the sensor comprises an air flow sensor.

4. The device of claim 3, wherein the air flow sensor can measure air flow in both inspiratory and expiratory directions.

5. The device of claim 3, wherein the air flow velocity sensor comprises a hot-wire anemometer.

6. The device of claim 3, wherein the air flow sensor comprises a thermistor.

7. The device of claim 1, wherein the sensor comprises a pH sensor.

8. The device of claim 1, wherein the catheter further comprises an occluding balloon.

9. The device of claim 1, wherein the sensor relays data to an external device.

10. A method of treating emphysema, the method comprising:
advancing a sensor to a first reference site located in a lung;
measuring oxygen concentration from the first reference site during at least one patient breathing cycle;
advancing the sensor to one or more lung segments located more distally than the first reference site and measuring oxygen concentration from the one or more lung segments during at least one patient breathing cycle;
correlating the oxygen concentration measured at the one or more lung segments with the oxygen concentration measured at the reference site to determine lung segments with poor oxygen absorption; and
placing one or more treatment devices in the lung segments identified to have poor oxygen absorption.

11. The method of claim 10, wherein the treatment device comprises a one-way valve.

12. The method of claim 10, wherein the oxygen concentration is measured at several lung segments and the lung segments are ranked in order of oxygen absorption, and treatment devices are placed in the distal lung segments where the oxygen absorption is below a predetermined cut-off value.

13. The method of claim 10, wherein the oxygen absorption at the one or more distal lung segments is represented graphically for analysis by an operator.

14. A method of treating air leaks in a lung, the method comprising inserting a gas sensor into a chest tube inserted at least partially into a patient's lung, and further comprising:
isolating a distal lung passageway;
pressurizing the isolated distal lung passageway with a gas;
monitoring a gas concentration with the gas sensor; and
if the gas concentration increases, occluding the distal lung passageway, or, if the gas concentration does not increase, repeating the procedure at a different distal lung passageway.

15. The method of claim 14, wherein the gas is oxygen.

16. The method of claim 14, wherein the lung passageway is occluded with a one-way valve.

17. A method of detecting collateral ventilation in a lung, comprising:
inserting an air flow sensor into a lung passageway;
detecting air flow velocity using the air flow sensor in the lung passageway during inspiration; and,
detecting air flow velocity using the air flow sensor in the lung passageway during expiration, wherein if the air flow velocity detected during inspiration is greater than the air flow velocity detected during expiration, the passageway is determined to be feeding collateral passageways, and is treated.

18. The method of claim 17, wherein the treatment comprises a one-way valve.

19. A method of treating an aerobic bacterial infection in a lung, comprising,
obtaining a reference oxygen depletion rate;
advancing a catheter comprising an oxygen sensor and an occluding balloon to a lung segment suspected to be infected;
occluding the lung segment suspected to be infected with the occluding balloon;
measuring the oxygen depletion rate in the lung segment suspected to be infected; and,
initiating treatment if the oxygen depletion rate in the lung segment suspected to be infected is greater than an a reference oxygen depletion rate.

20. The method of claim 19, wherein the treatment comprises occluding a lung segment with a one-way valve.

21. The method of claim 19, wherein the reference oxygen depletion rate is obtained from a segment of the lung known to be uninfected.

22. The method of claim 19, wherein the reference oxygen depletion rate is obtained from a standardized data source.

23. A method of treating lung tumors in a patient breathing an oxygen-enriched gas mixture, comprising
advancing a catheter comprising an oxygen sensor to a lung tumor site;
advancing a combustion-causing tumor treatment device to the lung tumor site;
monitoring the oxygen concentration at the lung tumor site; and
initiating treatment at the lung tumor site with the tumor treatment device while the oxygen concentration at the lung tumor site is below a predetermined value.
